(19) United States
(12) Patent Application Publication
(10) Pub. No.: US 2015/0238139 A1
(43) Pub. Date: Aug. 27, 2015

(54) WEARABLE DEVICES AND INTERFACES FOR MANAGING ACTIVITIES RESPONSIVE TO PHYSIOLOGICAL EFFECTS OF CONSUMABLES

(71) Applicants: Aza Benjamin Blum Raskin, San Francisco, CA (US); Thomas Edward Coates, San Francisco, CA (US); Matthew Simon Biddulph, San Francisco, CA (US)

(72) Inventors: Aza Benjamin Blum Raskin, San Francisco, CA (US); Thomas Edward Coates, San Francisco, CA (US); Matthew Simon Biddulph, San Francisco, CA (US)

(73) Assignee: AliphCom, San Francisco, CA (US)

(21) Appl. No.: 14/187,317

(22) Filed: Feb. 23, 2014

(51) Int. Cl. A61B 5/00; A61B 5/145; A61B 5/11

(52) U.S. Cl. CPC A61B 5/4866; A61B 5/7275; A61B 5/7282; A61B 5/4815; A61B 5/4809; A61B 5/1118; A61B 5/14546; A61B 5/743; A61B 5/7475; A61B 5/681; A61B 5/0022

(57) ABSTRACT

Embodiments of the invention relate generally to electrical and electronic hardware, computer software, wired and wireless network communications, and wearable computing devices for facilitating health and wellness-related information. More specifically, disclosed are wearable devices, interfaces, and methods to manage activities, including a sleep activity, responsive to physiological effect of a consumable. In one embodiment, a method includes receiving data representing an indication of consumption, and identifying an amount of a consumable portion. Also, the method includes receiving data representing a time of an event, and aggregating amounts of consumable portions to form an aggregated amount. A modified amount of the aggregated amount of the consumable portions can be determined, for example, as a function of a dissipation rate. The method can include predicting the modified amount at the time of the event, and causing presentation of a time at which a predicted amount conforms to the threshold value.

### CROSS-RELATED APPLICATIONS

This application incorporates herein by reference the following applications or patents: U.S. Pat. No. 8,445,274 that issued on May 21, 2013, is hereby incorporated by reference for all purposes. Also, U.S. patent application Ser. No. 13/802,305 filed on Mar. 13, 2013 with an Attorney Docket No. ALI-267 and U.S. patent application Ser. No. 13/802,319 filed on Mar. 13, 2013 with an Attorney Docket No. ALI-268 are both hereby incorporated by reference for all purposes. Further, U.S. patent application Ser. No. 13/433,204, filed on Mar. 28, 2012 having Attorney Docket No. ALI-013CIP1, U.S. patent application Ser. No. 13/433,208, filed Mar. 28, 2012 having Attorney Docket No. ALI-013CIP2, U.S. patent application Ser. No. 13/433,208, filed Mar. 28, 2012 having Attorney Docket No. ALI-013CIP3, U.S. patent application Ser. No. 13/454,040, filed Apr. 23, 2012 having Attorney Docket No. ALI-013CIP1CIP1, and U.S. patent application Ser. No. 13/627,997, filed Sep. 26, 2012 having Attorney Docket No. ALI-100 are all incorporated herein by reference for all purposes.

### FIELD

Embodiments of the invention relate generally to electrical and electronic hardware, computer software, wired and wireless network communications, and wearable computing devices for facilitating health and wellness-related information. More specifically, disclosed are wearable devices, interfaces, and methods to manage activities, including a sleep activity, responsive to physiological effect of a consumable.

### BACKGROUND

Devices and techniques to gather monitor physiological information regarding the physiological effects of a consumable, such as a level of metabolized substance upon a person, in view of activities performed by the person. Conventional techniques, while often available and functional, are not well-suited to capture such information other than by using conventional data capture devices. Conventional devices, often clinical in nature (e.g., laboratory equipment), typically lack capabilities to capture, analyze, communicate, or use physiological-related data in-situ (e.g., during normal activities) that otherwise might provide a contextually-meaningful, comprehensive, and efficient manner, such as during the day-to-day activities of a user, including high impact and strenuous activities or during sleep. While functional, the traditional devices and solutions to collecting physiological effects of consumables are not well-suited for active participants in sports or over the course of over a period of time, such as one or more days, weeks or months.

Thus, what is needed is a solution for data capture devices, such as for wearable devices, to provide interfaces and methods to monitor and/or manage physiological effects of a consumable on an individual without the limitations of conventional techniques.

### BRIEF DESCRIPTION OF THE DRAWINGS

Various embodiments or examples (“examples”) of the invention are disclosed in the following detailed description and the accompanying drawings:

FIG. 1 illustrates an example interface and controller to convey physiological information relating to a consumable and the effects thereof, according to some embodiments;

FIG. 2 illustrates an example interface and controller to convey additional information relating to a consumable and the effects thereof, according to some embodiments;

FIG. 3A depicts an example of a device including a touchscreen configured to facilitate monitoring and management of physiological effects responsive to a consumable in accordance with at least some embodiments;

FIG. 3B illustrates an example of an interface for implementing various techniques describe herein, according to various embodiments;

FIG. 4 is an example flow diagram for generating an interface to monitor and/or manage activities responsive to physiological effects of the consumable, according to some embodiments;

FIG. 5 is a diagram depicting a consumable controller, according to some embodiments;

FIGS. 6A and 6B depict a controller configured to correlate amounts of consumable portions against an activity, such as sleep, according to various embodiments;

FIG. 7 depicts a controller including a sleep manager, according to some embodiments;

FIGS. 8A and 8B depict a notification generator configured to generate notifications as a function of correlation of data, according to some examples;

FIG. 9 depicts a notification generator configured to generate reports as a function of correlation of data, according to some examples;

FIGS. 10A and 10B depict a notification generator configured to generate notifications as a function of correlation of data, according to some examples;

FIG. 11 is a flowchart for correlating sleep characteristics and consumption of a consumable, such as caffeine, according to some embodiments;

FIG. 12 is a block diagram depicting generation of a notification to avoid symptoms associated with withdrawal, according to some examples; and

FIG. 13 illustrates an exemplary computing platform in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

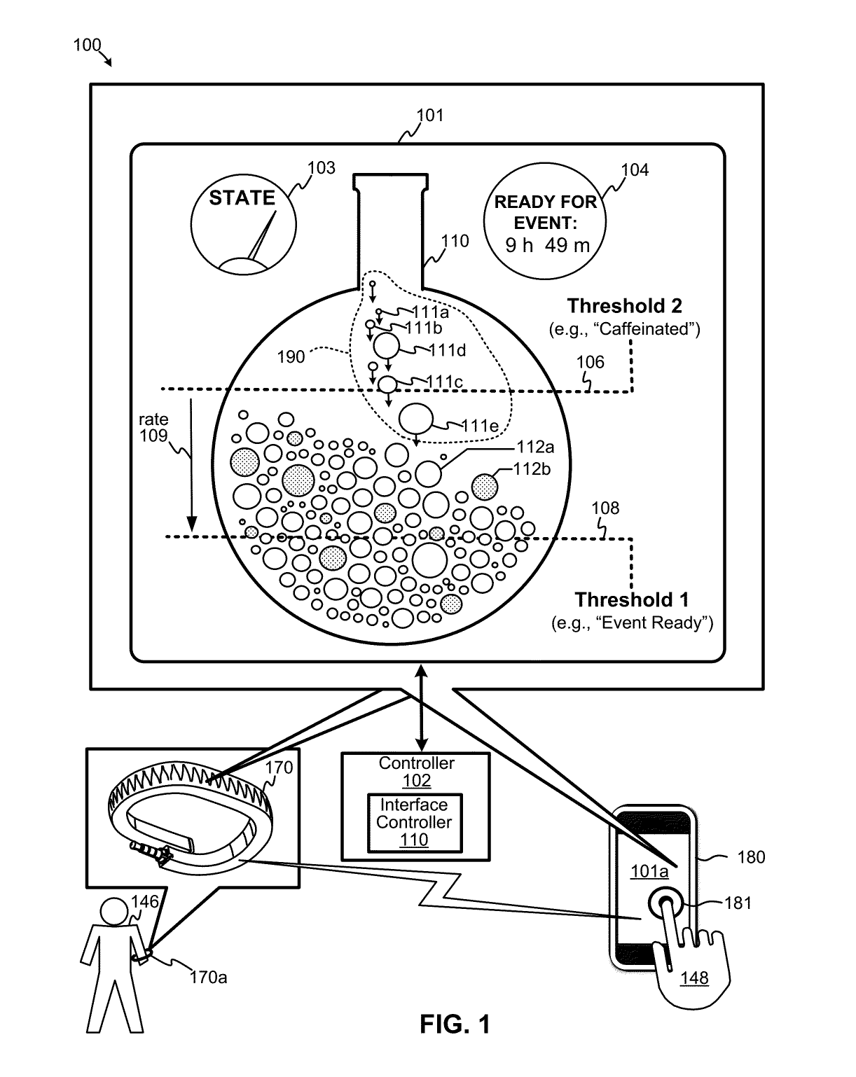

FIG. 1 illustrates an example interface and controller to convey physiological information relating to a consumable and the effects thereof, according to some embodiments. Diagram 100 depicts a controller 102 configured to determine an amount of a consumable or a portion of the consumable, such as an amount of caffeine, for determining a state of a user that is metabolizing the portion of the consumable. Controller 102 also is configured to predict an amount of consumable portion at some time, based on a rate of dissipation (e.g., rate of metabolization) of the consumable portion. Further, controller 102 is configured to monitor amounts of the consumable portion at different time points and characteristics of one or more activities. Controller 102 is configured to correlate the amounts of consumable portion to the characteristics of the one or more activities to determine, for example, the physiological effects of the consumable portion upon an individual's performance of an activity, such as a sleep activity. Diagram 100 also depicts controller 102 including an interface controller 110 is configured to generate an interface 101, which can be configured to receive signals, such as data signals representing user input requests. Interface 101 also can be configured to present or convey an accumulated amount of consumable portions that are, for example, yet to be metabolized. As such, the accumulated amount of consumable portions can affect the user physiologically, whereby an activity performed by a user, in turn, may be influenced (e.g., positively or negatively) by the consumption of additional amounts of consumable portions (whether consumed at present or at a future-determined time).

According to some examples, controller 102 (or portions thereof) can be disposed in and/or distributed over a wearable device 170 and/or mobile device 180. As such, interface controller 110 can generate interface 101 as interface 101a of mobile device 180. While diagram 100 depicts an example of wearable device 170 being worn by user 146 as wearable device 170a, wearable device 170 also refers to devices that can be carried or attached to clothing/accessory worn by a user 146. Controller 102, according to some embodiments, is configured to identify an amount of a portion of the consumable, such as an amount of units of caffeine (e.g., expressed in units of milligrams, or mg) and to receive data representing a time of an event. The time of the event may coincide with an expected performance of a certain future activity, such as exercising or engaging in a sleep activity.

As shown in FIG. 1, interface controller 110 is configured to generate interface 101, which is shown to include a vessel, such as a flask 110, into which amounts of consumable portions are added (e.g., responsive to a user drinking a cup of coffee) and aggregated (e.g., combining yet-to-be dissipated amounts of caffeine). Controller 102 is configured to determine a first threshold 108 that specifies a sufficient amount of caffeine below which the user is “ready” to engage an event, such sleep. That is, first threshold 108 specifies an amount of consumable portions that likely provide optimal performance of an activity by user, such as having quality sleep, that otherwise might be impacted by higher levels of consumable portions above first threshold 108. Further, controller 102 is configured to determine a second threshold 106 above which a user's performance or physiological state may be influenced (e.g., negatively) by increased a bounce a consumable portions above second threshold 106. For example, second threshold 106 can represent 400 mg of caffeine (or greater), whereas first threshold 108 can represent 100 mg of caffeine (or less). Responsive to establishing thresholds 106 and 108, interface controller 110 can generate data signals to cause presentation of representations (e.g., as dotted lines) of thresholds 106 and 108 in interface 101.

Next, consider that controller 102 is configured to add amounts of caffeine to accumulated amounts of caffeine of flask 110 responsive to user inputs. As used herein, at least in some examples, “cumulative amounts” of consumable portions can refer to previously consumed portions that are yet to be dissipated. Interface controller 110 can detect an amount of consumable portion (e.g., to be consumed by a user), for example, response to a user 148 interacting with interface 101a to select an input 181, which generates data signals representing an amount of a consumable portion (e.g., 200 mg of caffeine) associated with consuming a strongly-brewed cup of coffee (the coffee being a consumable). Responsive to the generated data signals, controller 102 is configured to determine a number of units that are divided from the added amount of the consumable portion. In some cases, controller 102 and/or interface controller 110 generates data representing a representation of a number of units of the aggregated amount of the portions of the consumable, the number of units being shown in FIG. 1 is having different sizes in flask 110. Further, controller 102 and/or interface controller 110 can cause presentation of the representation (e.g., differently-sized shapes) of the number of units. Further, in accordance with a rate 109 at which the units of consumable portions participated, controller 102 and/or interface controller 110 can reduce the number of units as a function of the dissipation rate (e.g., rate 109) to form a reduced number of units of the aggregated amount in flask 110. Controller 102 and/or interface controller 110 then can cause presentation of a representation of the reduced number of units (i.e., fewer circles in flask 110).

As another example, consider that interface controller 110 generates a group 190 of different representations 111 for the differently-sized units, and then animates the different representations 111 with motion to appear as to drop through the neck (at top) of flask 110, under the influence of gravity, to aggregate with previously-disposed representations of units of consumable portions. To illustrate, consider that controller 102 determines that 50 mg of caffeine is consumed, and divides that amount to sub-portions or units that can be represented by different sizes to indicate different sub-amounts of caffeine. For example, first representations 111d and 111e can represent 15 mg of caffeine, a second representation 111c can represent 10 mg of caffeine, a third representation 111b can represent 5 mg of caffeine, and a fourth representation 111a can represent 2.5 milligrams of caffeine, and the like. Therefore, if controller 102 determines amounts of caffeine that are dissipated per unit time, controller 102 can cause interface controller 110 to remove from interface 101 a combination of units/representations to match a dissipated amount.

Controller 102 is further configured to aggregate the amount of the portion of the consumable (e.g., the amount of caffeine associated with group 190) and a cumulative amount of the portions of the consumable to form an aggregated amount of the portions of the consumable (e.g., a total amount of caffeine that is yet to be dissipated at the time group 190 was added). As a function of the dissipation rate, controller 102 determines a modified amount of the aggregated amount, which can be a reduced amount. Controller 102 can also form a predicted amount by predicting the modified amount at the time of the event. To illustrate, consider that prior to adding group 190, the cumulative amount of dissipated caffeine, relative to threshold 109, was to dissipate and five hours and 30 minutes (5 h 30 m). But the additional consumption of 200 mg of caffeine associated with group 190 causes the aggregated amount to increase, which, in turn, causes an increase in time for the aggregated amount to reach threshold 108. Therefore, if the user was targeting an optimal level of caffeine to achieve unimpeded sleep, that user would experience a longer time to reach threshold 108. In this example, consider that the 200 mg of caffeine boosted the time to reach threshold 108 is nine hours and 49 minutes (9 h 49 m). Responsive to this determination, controller 102 and/or interface controller 110 can be configured to cause rates of gun this done presentation of a representation 104 of a time at which the predicted amount conforms to (e.g., reaches) threshold value 108.

In some other examples, controller 102 can generate data representing a representation 103 of a state of the user as a function of a modified amount of caffeine as it dissipates over time. Interface controller 110 can generate data signals to cause presentation of representation 103 of the state of the user on interface 101. In some examples, different states of a user can correspond to different ranges of amounts of caffeine that are yet to metabolize. For example, the state of “ready for bed” can be ascribed to user whose level of caffeine is below threshold 108. As another example, a state of “calm” can be ascribed to user whose level of caffeine is 100 mg above threshold 108. In yet another example, a state of being “wired” can be described to user whose level of caffeine is at or above threshold 106. In some cases, controller 102 is configured to generate data representing a notification, which is configured to cause the predicted amount of caffeine or other consumable portions to conform or meet threshold 108 at the time of the event (e.g., a targeted sleep time). For example, a notification can inform the user that their sleep might be disrupted or less than optimal should they choose to proceed to drink a couple coffee at a later time of day, such as 7 PM.

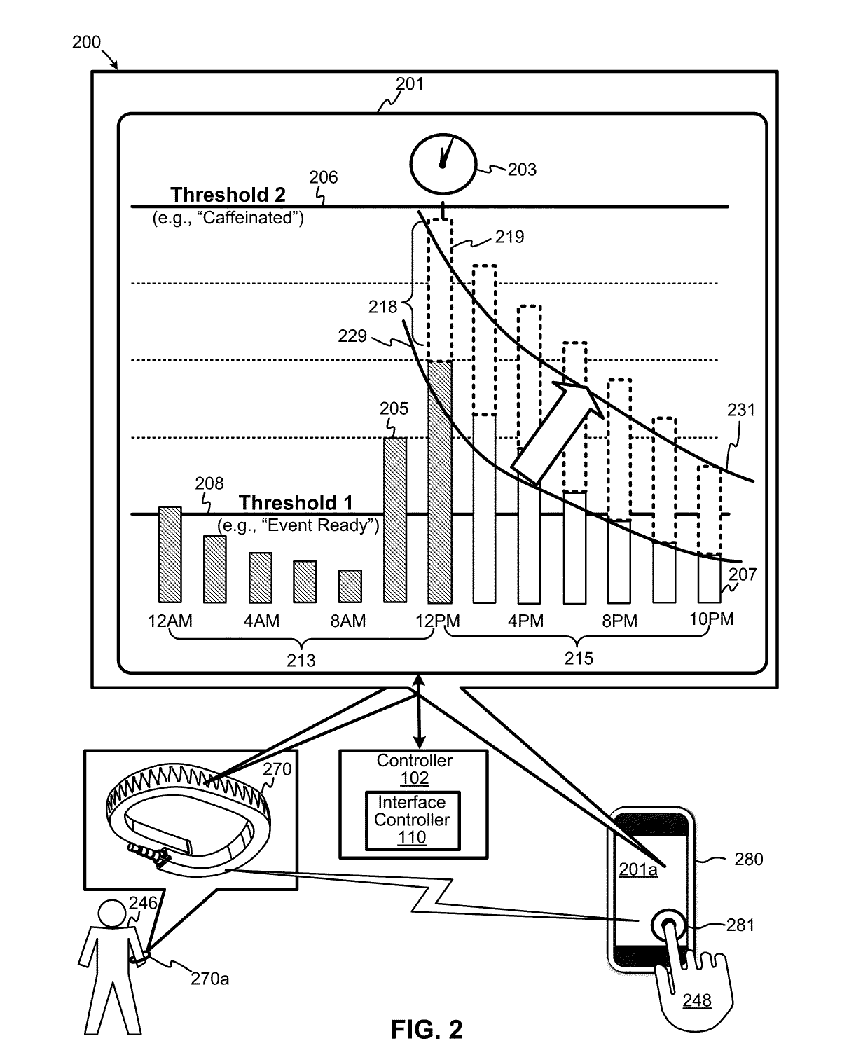

FIG. 2 illustrates an example interface and controller to convey additional information relating to a consumable and the effects thereof, according to some embodiments. Diagram 200 depicts controller 102 configured to determine the amount of a consumable portion, such as an amount of caffeine, to predict an amount of consumable portion at some time, based on a rate of dissipation (e.g., rate of metabolization) and to correlate the amounts of consumable portion to the characteristics of the one or more activities, such as sleep to determine whether an individual's performance may be impacted or otherwise influenced by the amount of caffeine in the body of the user. Further to the examples of FIG. 1, controller 102 (or portions thereof) can be disposed in and/or distributed over a wearable device 270 and/or mobile device 280. An example of wearable device 270 is worn as wearable device 270a by user 246. As such, interface controller 110 can generate interface 201 as interface 201a of mobile device 280. Controller 102, according to some embodiments, is configured to identify an amount of a portion of the consumable, such as an amount of units of caffeine (e.g., expressed in units of milligrams, or mg) and to receive data representing a time of an event. The time of the event may coincide with an expected performance of a certain future activity, such as engaging in a sleep activity.

As shown in diagram 200, interface controller 110 is configured to depict levels 215 of caffeine at different points in time 213 in the past, and to predict levels 207 (shown with cross-hatch) of caffeine at different points in time 215 in the future. Similar to FIG. 1, interface 201 includes a first threshold 208 and a second threshold 206, which are similar in structure and/or functionality as set forth in similarly-described elements in FIG. 1. By contrast, interface 201 includes a reference representation 203 indicating approximate time or present time at which a user is considering or is consuming a consumable portion. Thus, representation 203 can specify a time at which the consumable, such as coffee, is consumed along with a consumable portion, such as caffeine. Further, interface 201 depicts a relationship 229 between time and modified amounts of caffeine levels at different time points after which some of the caffeine is dissipated. In some cases, relationship 229 is equivalent or approximate to a half-life curve or relationship in which a duration of time can be predicted in which the level of caffeine is dissipated by one-half.

As shown, if the user as a target time to fall asleep at 10 PM, that user likely will meet that goal if no further caffeine is consumed as relationship 229 is below threshold 208 at 10 PM. However, consider that the user desires a cup of coffee, and user 248 selects input 281 on interface 201a, and, in response, controller 102 determines an additional amount 218 of caffeine shown in dashed lines 219 for the present time, and in subsequent points in time. While the user need not have consumed the coffee at this time, the user can make a choice as to whether to consume or refrain from drinking coffee. Controller 102 is configured to generate data representing different values of the modified amounts of caffeine at different time points, such as every two hours. Interface controller 110 is configured to cause presentation of the representations for the different values at different times, as shown. Further, interface controller 110 is configured to cause the shifted relationship 231 to be presented to the user via interface 201. From this view, the user confirms that the predicted level of caffeine at 10 PM exceeds threshold 208, and, thus can make a decision whether to drink all, some, or none of the coffee to reduce the level of caffeine and user's blood stream at 10 PM.

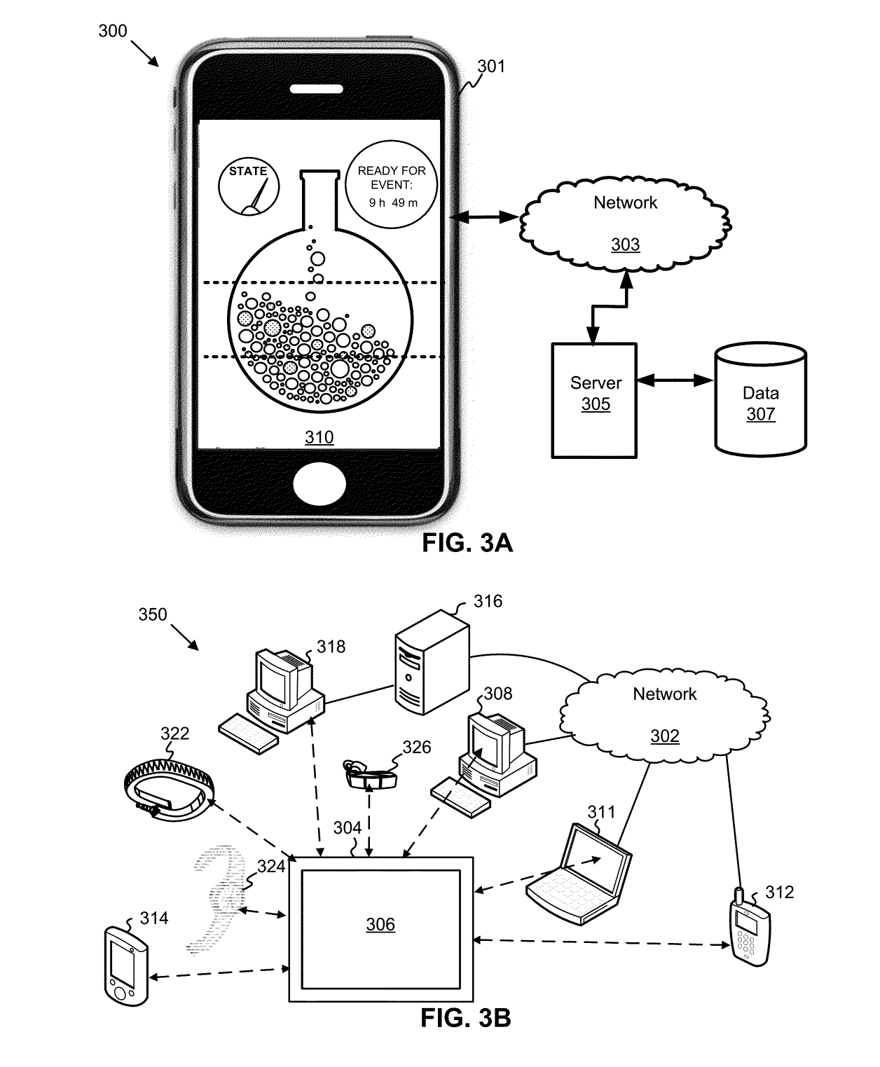

FIG. 3A depicts an example of a device including a touchscreen configured to facilitate monitoring and management of physiological effects responsive to a consumable in accordance with at least some embodiments. Device 301 includes logic configured to present in a touchscreen interface 310 graphic representations of levels consumable portions and related effects on an upcoming activity. In some embodiments, device 301 is configured to exchange data via network 303 with either server 305, to access logic, or repository (“data’) 307 to access data. According to various embodiments, server 305 can provide logic to generate correlations between data representing amounts of caffeine and characteristics of sleep for data (e.g., historic trend data) stored in repository 307. Further, any logic, structure and/or function described herein, or variants thereof, can be disposed either in device 301 or in the combination of server 305 and repository 307, or distributed in both.

FIG. 3B illustrates an example of an interface for implementing various techniques describe herein, according to various embodiments. Here, computing device 300 is coupled to a network 302, a display environment 304, an interface 306, which can be presented on devices such as computer 308, notebook computer (“notebook” or “laptop”) 311, mobile or smart phone 312, personal digital assistant (“PDA”) or tablet 314, server 316, and administrator computer 318. Further, an interface and/or logic for implementing any of the functionalities described herein can be implemented in a wear able device 322, a watch 325 (or watchband), or a headset 326. In other examples, the number and type of devices can be varied and are not limited to those shown and described. In some examples, interface 306 can be an interface for an application, an application programming interface (“API”), a web browsing program, an Internet content portal, a client or desktop application for any purpose, or the like. In one embodiment, interface 306 can include any number and/or any type of display environments, such as CRT and LCD displays, including touch-sensitive screens. Note that the above-described system and elements can be varied and are not limited to the descriptions or examples provided.

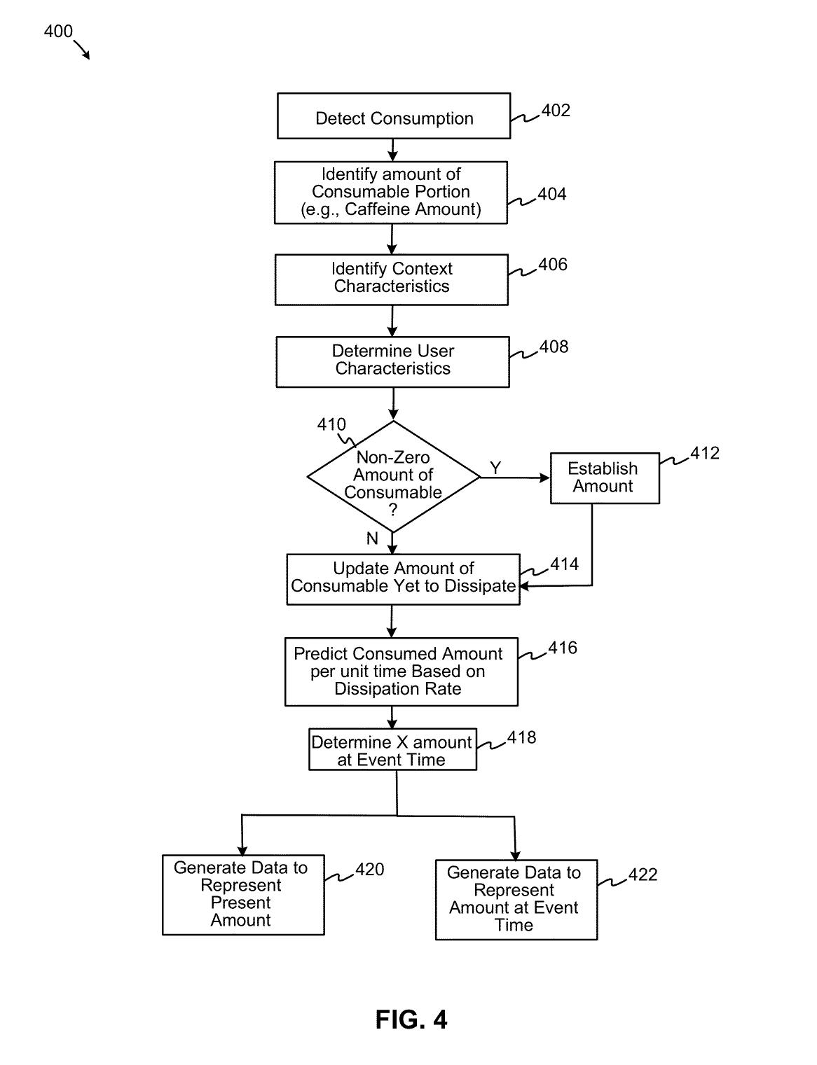

FIG. 4 is an example flow diagram for generating an interface to monitor and/or manage activities responsive to physiological effects of the consumable, according to some embodiments. At 402, flow 400 provides for the detection of a consumable. In some embodiments, the user interacts with an interface from which a controller determines that an amount of a consumable portion has been consumed. In other examples, motion data from a sensor is configured to determine a motion pattern that matches an activity of drinking (e.g., a hand-arm combination travels a path to a location at which a user's mouth is typically located and is accompanied with a tilting motion that is detected as liquid passes from a container, under the influence of gravity, into the user's mouth). At 404, an amount of a consumable portion is identified. For example, an amount of caffeine consumed may be identified. At 406, context characteristics are identified. For example, one context characteristic is a time at which caffeine is consumed, or is a time at which a non-dissipated aggregated caffeine level exists. At 408, user characteristics are determined. Different physical and physiological characteristics of a user, as well as various conditions or states of a user, can influence a rate at which the consumable portion, such as caffeine, is metabolized, or can exacerbate the effects of an otherwise normal level of caffeine (e.g., persons with anxiety issues typically exhibit more jittery physiological effects with smaller amounts of caffeine). At 410, a determination is made as to whether an aggregated amount of consumable portions is nonzero (e.g., whether there is caffeine detected in a user's system). If yes, flow 400 moves to 412 at which an initial level or amount of caffeine is established. Otherwise, flow 400 moves to 414, at which an added amount of caffeine is used to update an aggregated amount that is yet to dissipate. At 416, a consumed amount of consumable portions is predicted per unit time, based on a dissipation rate, whereby the consumed amount is predicted to be modified as the body of the user metabolizes the consumable portions, including caffeine. At 418, a predicted aggregated amount, X, of consumable portions is determined for a time that coincides with an event, such as a sleep activity. At 420, data representing a present amount of, for example, caffeine can be generated, and at 422, data representing a predicted amount of, for example, caffeine at the time of the event can also be generated.

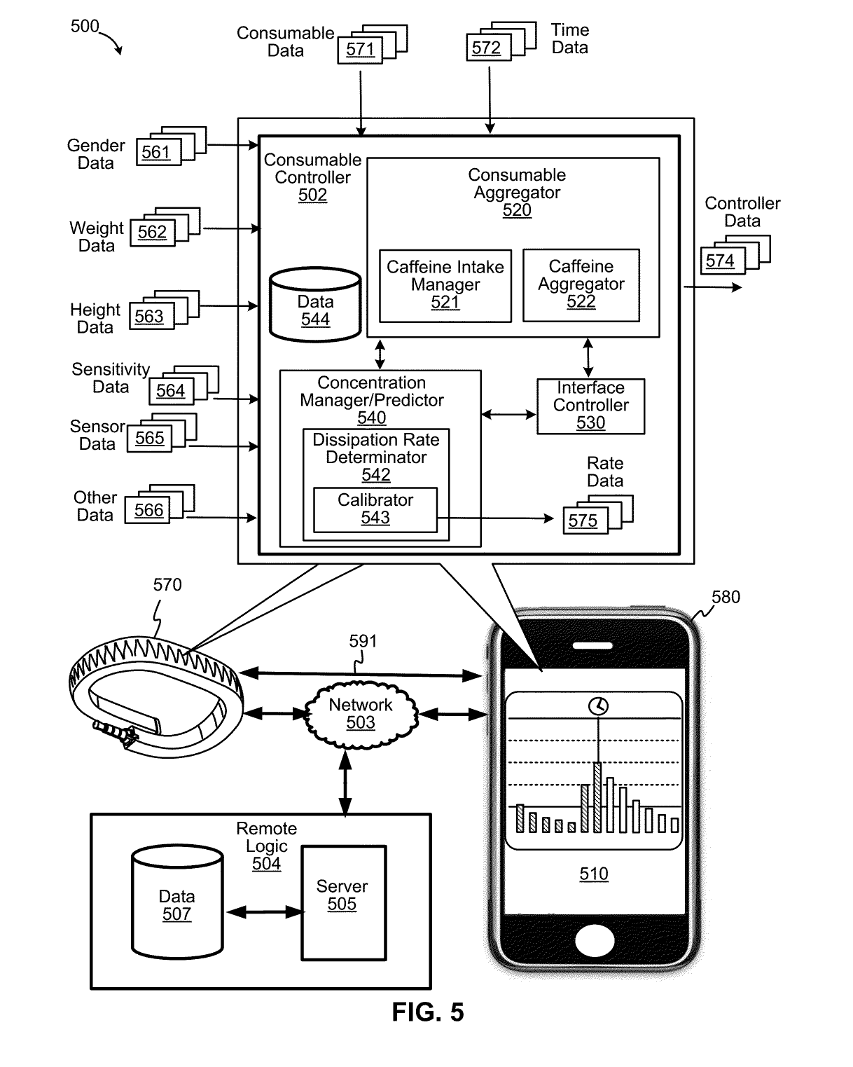

FIG. 5 is a diagram depicting a consumable controller, according to some embodiments. As shown, consumable controller 502 includes a consumable aggregator 520, a concentration manager/predictor 540, a repository 544 (e.g., memory), and an interface controller 530. Consumable controller 502 (or portions thereof) can be disposed in a wearable device 570 and/or mobile device 580, either of which can communicate with the other via a direct communications link 591 or via a network 503. Consumable controller 502 also is configured to receive various amounts of data, and is further configured to access remote logic 504, which includes a server device 505 and the repository 507. Remote logic 504 can perform various analytics and correlations between a user's caffeine levels and activities, such as sleep, as well as multiple activities. Remote logic 504 can perform various correlations against data for groups of other users that are similarly-situated. As shown, consumable controller 502 receives consumable data 571 and time data 572, which can describe a time at which the consumable portion is ingested. Consumable data 571 can include data describing at least a consumable portion (e.g., caffeine, or the like), and can also include data describing some or all constituent portions of a consumable. Consumable data 571 can include an indication of consumption of a consumable.

For example, coffee is a consumable that not only includes caffeine but also includes water as another consumable portion. Should there be cream in the coffee, the proteins and other constituent ingredients of the cream can be also referred to as consumable portions. According to various embodiments, the various structures and functionalities described herein are not limited to monitoring and managing activities based on caffeine levels. Rather the various structures and functionalities described herein can be applied to any consumable item, including medicines, drugs, food, and nutrients, micronutrients, macronutrients, and the like. Thus, the term “consumable portion” is intended to be inclusive and is not limited to caffeine.

Further, consumable controller also receives data representing a gender 561, data representing a weight 562, data representing a height 563, sensitivity data 564 representing a degree of sensitivity to a consumable portion (e.g., sensitivity to caffeine), sensor data 565 originating from one or more sensors (e.g., from sensors disposed in wearable device 570, mobile device 580, and the like), and other data 566 can include any kind or type of data they can be used to determine physiological effects of a consumable upon the user during performance of an activity, such as sleep. Examples of sensor data 565 and other data 566, as well as examples of wearable device 570 are disclosed in U.S. Pat. No. 8,445,274 that issued on May 21, 2013, whereby U.S. Pat. No. 8,445,274 is incorporated herein by reference for all purposes. Other data 566 also can include data representing a target bedtime at which the user wishes to begin a sleep activity.

Diagram 500 also depicts that consumable aggregator 520 includes a caffeine intake manager 521 and a caffeine aggregator 522. In some examples, consumable aggregator 520 is configured to receive consumable data 571 to extract the amounts and types of consumable portions that have been, or will be, consumed. In some examples, consumable aggregator 520 is configured to determine a level or amount of each consumable portion over, for example, a duration of time. While consumable aggregator 520 can include a variety of intake managers, similar to caffeine intake manager 521, and a variety of aggregators, similar to caffeine aggregator 522, for other types of consumable portions, diagram 500 depicts only caffeine intake manager 521 and caffeine aggregator 522 for purposes of discussion. Caffeine intake manager 521 is configured to determine an amount of caffeine (as well as consumable in which caffeine was included) and a time at which the amount of caffeine was consumed. Caffeine aggregator 522 configured to aggregate added caffeine levels to cumulative caffeine levels due to previous consumption of caffeine. Caffeine aggregator 522 transmits data representing an amount of aggregated levels of caffeine to, for example, interface controller 530 to cause a representation of the amount of aggregated caffeine levels via an interface 510 to a user. Interface controller 530 can transmit data representing graphical representations for displaying a screen as part of data 574, which can include other types of data generated by consumable controller 502.

Concentration manager/predictor 540 is shown to include a dissipation rate determinator 542, which, in turn, includes an optional calibrator 543. Concentration manager/predictor 540 is configured to determine a present concentration of, for example, caffeine, and predicted levels of caffeine at different points in time in the future based on a rate at which caffeine dissipates. Dissipation rate determinator 542 is configured to determine the rate at which caffeine (or any other consumable item) dissipates. In some examples, a dissipation rate for caffeine can be a function of gender, weight, height, and relative sensitivity to caffeine. As such, dissipation rate determinator 542 receives data 561, 562, 563, and 564 to determine a dissipation rate based on at least the aforementioned data. For example, a dissipation rate can be established as having a half-life based on physical and/or physiological characteristics of the user, among other things. For example, females tend to metabolize caffeine more quickly than males. Further, other data 566 can include data representing whether the user is a smoker or whether the user is consuming oral contraceptives. In particular, smokers tend to metabolize caffeine more quickly than nonsmokers, whereas women taking oral contraceptives may metabolize caffeine at a slower rate. Calibrator 543 is configured to receive such data, as well as any other data that might assist in calibrating a dissipation rate by generating data 575 representing an updated rate. In some examples, calibrator 543 can automatically modify a sensitivity level of a user to caffeine based on archived data relating to caffeine and other activities. In one embodiment, a user can select an input that generates data specifying whether the user is either not sensitive to caffeine, somewhat sensitive to caffeine, or very sensitive to caffeine. Such selections can be used to calibrate a dissipation rate (i.e., by speeding it up or slowing it down).

In some embodiments, wearable device 570 can be in communication (e.g., wired or wirelessly) with a mobile device 580, such as a mobile phone or computing device. In some cases, mobile device 580, or any networked computing device (not shown) in communication with wearable device 570 or mobile device 580, can provide at least some of the structures and/or functions of any of the features described herein. As depicted in FIG. 5 and other figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 5 (or any other figure) can represent one or more algorithms. Or, at least one of the elements can represent a portion of logic including a portion of hardware configured to provide constituent structures and/or functionalities.

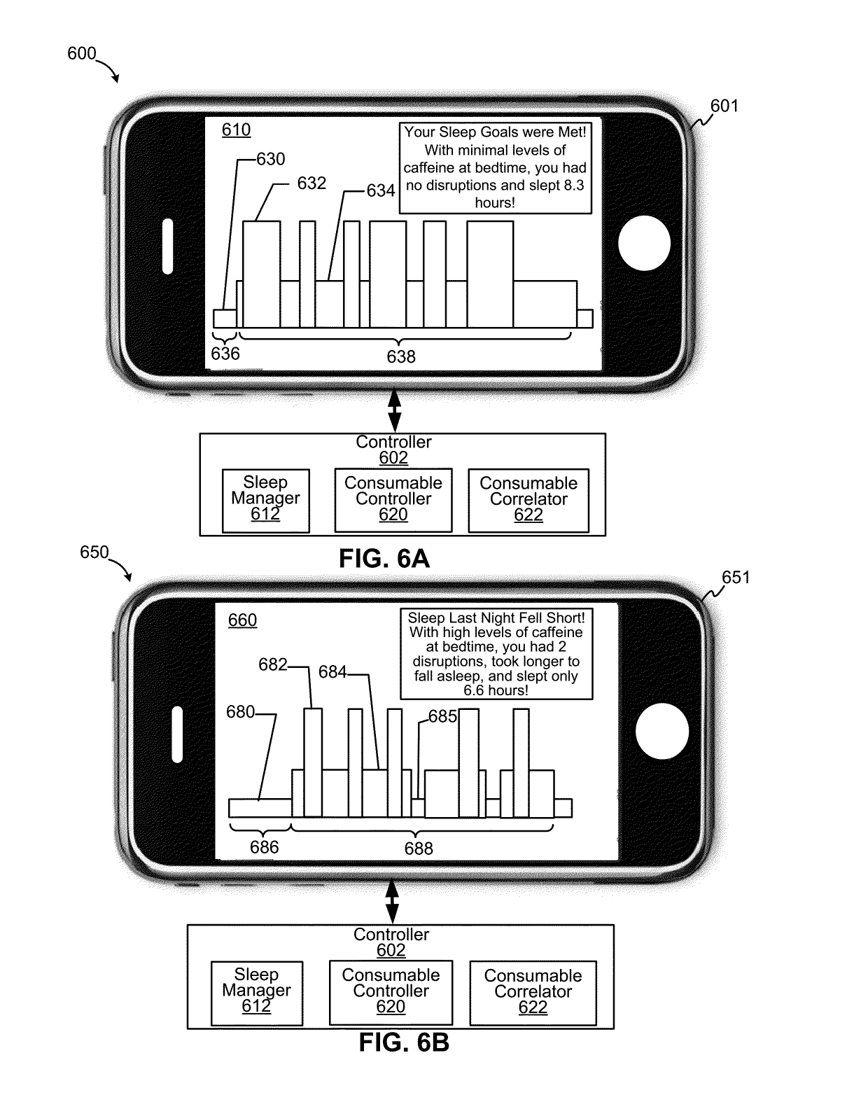

For example, controller 102 of FIG. 1, controller 602 of FIG. 6, and consumable controller 502 of FIG. 5 and any of its one or more components, such as consumable aggregator 520, concentration manager/predictor 540, interface controller 530, and repository 544, can be implemented in one or more computing devices (i.e., any mobile computing device, such as a wearable device or mobile phone, whether worn or carried) that include one or more processors configured to execute one or more algorithms in memory. Thus, at least some of the elements in FIG. 5 (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, controller 102 of FIG. 1, controller 602 of FIG. 6, and consumable controller 502 and any of its one or more components, such as consumable aggregator 520, concentration manager/predictor 540, interface controller 530, and repository 544, can be implemented in one or more computing devices that include one or more circuits. Thus, at least one of the elements in FIG. 5 (or any other figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

FIGS. 6A and 6B depict a controller configured to correlate amounts of consumable portions against an activity, such as sleep, according to various embodiments. Diagram 600 of FIG. 6A depicts a controller 602 configured to interact with interface 610, whereby controller 602 may be disposed in computing device 601. Controller 602 is shown to include a sleep manager 612, a consumable controller 620, and a consumable correlator 622. In at least some examples, sleep manager 612 is configured to monitor, characterize, determine, analyze, and identify characteristics of an activity, such as sleep. For example, sleep manager 612 can determine a length of time 636 before an onset of sleep. During the length of time 636, the user is awake and not yet asleep. Another characteristic of sleep, as determined by sleep manager 612, is a duration of sleep 638 between wakeful states 630. Other characteristics of sleep includes a number of times a user enters deep sleep 632, which can include REM sleep in some examples, a length of time individually for each portion of the sleep cycle in deep sleep 632, and a cumulative amount of time that a user spends and deep sleep 632. These characteristics for light sleep 634 are also monitored and identified similar to as described for deep sleep 632. Also, sleep manager 612 tracks the number and duration of disrupted sleep (e.g., period of wakefulness during sleep), as well as amounts of motion that is beyond expected ranges of motion (e.g., motion due to nightmares, stress, pacing the room, excessive bathroom usage, etc.).

Examples of one or more sleep managers or equivalent structures and/or functionalities are described in U.S. Pat. No. 8,445,274 that issued on May 21, 2013, whereby U.S. Pat. No. 8,445,274 is incorporated herein by reference for all purposes. Also, other examples are described in U.S. patent application Ser. No. 13/802,305 filed on Mar. 13, 2013 with an Attorney Docket No. ALI-267 and U.S. patent application Ser. No. 13/802,319 filed on Mar. 13, 2013 with an Attorney Docket No. ALI-268, both of which are incorporated herein for all purposes.

As described herein, consumable controller 620 is configured to determine amounts of consumable portion such as levels of caffeine. Consumable controller 620 is also configured to determine the amounts of caffeine at or during various times before, during, and after a sleep cycle. Consumable correlator 622 is configured to correlate characteristics of a consumable, such as caffeine, against the characteristics of an activity, such as a sleep activity, to determine whether there is no correlation, a positive correlation, or a negative correlation between or among the sets of data. Consumable correlator 622 can implement any of a variety of correlation techniques, cross-correlation techniques, statistical techniques, and the like to determine relationships and/or associations between sets of caffeine data and sets of sleep data.

In the example shown in FIG. 6A, controller 602 determined either a user had levels of caffeine below a threshold, or that caffeine levels for a user did not significantly affect sleep. However, diagram 650 of FIG. 6B illustrates in interface conveying information that a user's sleep cycle was suboptimal in view of, for example, high levels of caffeine at bedtime. As shown in interface 660, a user had a prolonged length of time 686 during which he or she was in a wakeful state 680. Further, the duration of sleep 688 was 6.6 hours, which was less than the user's goal. Also shown in interface 660, the user had two disruptive state 685 in which the user awoke during the sleep cycle. And the deep sleep 682 portions and the light sleep 684 portions are depicted as being shorter and fewer than those depicted in FIG. 6A. Therefore, suboptimal sleep can be monitored and predicted based on various decision makes during the day on consuming caffeine.

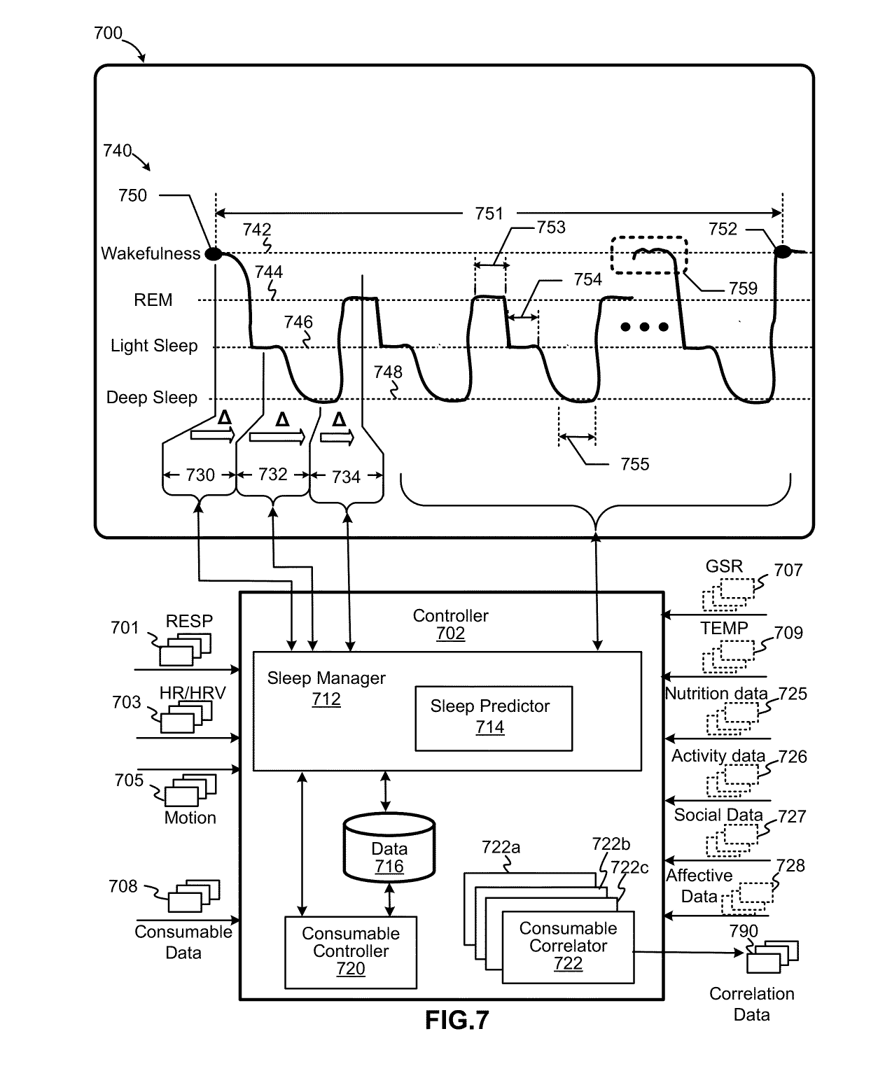

FIG. 7 depicts a controller including a sleep manager, according to some embodiments. As shown, FIG. 7 depicts a controller 702 including a sleep manager 712, a consumable controller 720, data repository 716 to store archived data (for correlating and statistical analysis, among other things), and a consumable correlator 722. Controller 702 is configured to receive physiological characteristic signals, such as data representing respiration rates (“RESP”) 701, heart rate (“HR”) 703 (or heart rate variability, HRV), motion-related data 705, and other physiological data such as optional skin conductance (“GSR”) 707 and optional temperature (“TEMP”) 709, among others. Further, controller 702 can also receive consumable data 708 includes information regarding types of consumables ingested (e.g., coffee or energy drink, with or without alcohol), amounts of caffeine consumption, times at which caffeine is consumed, etc.

In some cases, controller 702 can further receive nutrition data 725 that describes a number of meals or other ingested substances that might provide a correlation to either caffeine or sleep. For example, large amounts of caffeine may suppress an appetite, thereby delaying consumption of a meal, but once the effects of caffeine dissipates, a user may be more hungry than is normal. Also, activity data 726 can be received by controller 702, whereby activity data 726 (e.g., running data) may provide insight into a user's performance when correlated to caffeine or sleep. Controller 702 also receives social data 727 that describes various activities performed in groups of unknown users or in smaller groups of known users, such as friends. For example, correlations in caffeine intake are likely to be found if users spend a fair amount of time with those people who drink more coffee than average. Controller 702 also can receive affective data 728 that describes a person's mood, emotion, or stress-level. Such information can be used to determine whether caffeine is a contributor to a user's mood or anxiety level. Other correlators, such as correlators 722a to 722c, can be implemented to perform the above-described correlations among the various types as well as with physiological information based on respiration data 701, heart rate data 703, motion data 705, GSR data 707, and temperature data 709.

Sleep manager 712, which includes a sleep predictor 714, which is configured to determine physiological states of sleep, such as a sleep state or a wakefulness state in which the user is awake. Further, sleep manager 712 is configured to characterize, quantify, and/or describe various characteristics of sleep. Consumable correlator 722 is configured to correlate levels of caffeine associated with the user to the various characteristics of sleep to determine whether caffeine has at least some influence (e.g., including at least a weak correlation or a strong correlation) on sleep performance. Further, consumable correlator 722 is used to determine whether one or more sleep characteristics (or deviations therefrom) can be attributed to caffeine. Consumable correlator 722 further is configured to generate correlation data 790 includes various sets of data describing at least sleep characteristics and the degree of correlation with levels of caffeine.

As shown in diagram 740, a person who is sleeping passes through one or more sleep cycles over a duration 751 between a sleep start time 750 and sleep end time 752. There is a general reduction of motion when a person passes from a wakefulness state 742 into the stages of sleep, such as into light sleep 746 in duration 754. Motion indicative of “hypnic jerks” or involuntary muscle twitching motions typically occur during light sleep state 746. Consumable correlator 722, at least in some cases, can be configured to did distinguish these types of motions from excess of motions that might, for example, be due to excess caffeine. The person then passes into a deep sleep state 748, in which, a person has a decreased heart rate and body temperature, with the absence of voluntary muscle motions to confirm or establish that a user is in a deep sleep state. Collectively, the light sleep state and the deep sleep state can be described as non-REM sleep states. Further to diagram 740, the sleeping person then passes into an REM sleep state 744 for duration 753 during which muscles can be immobile.

According to some embodiments, sleep manager 712 is configured to determine a stage of sleep based on at least the heart rate and respiration rate. For example, sleep manager 712 can determine the regularity of the heart rate and respiration rate to determine the person is in a non-REM sleep state, and, thereby, can generate a signal indicating the stage of the sleep is a non-REM sleep states, such as light sleep or deep sleep states. During light sleep and deep sleep, a heart rate and/or the respiration rate of the user can be described as regular or without significant variability. Thus, the regularity of the heart rate and/or respiration rate can be used to determine physiological sleep state of the user. In some examples the regularity of the heart rate and/or the respiration rate can include any heart rate or respiration rate that varies by no more than 5%. In some other cases, the regularity of the heart rate and/or the respiration rate can vary by any amount up to 15%. These percentages are merely examples and are not intended to be limiting, and ordinarily skilled artisan will appreciate that the tolerances for regular heart rates and respiration rates may be based on user characteristics, such as age, level of fitness, gender and the like. Sleep manager 712 can use motion data 705 to confirm whether a user is in a light sleep state or a deep sleep state by detecting indicative amounts of motion, such as a portion of motion that is indicative of involuntary muscle twitching. Again, consumable correlator 722, at least in some cases, can be configured to did distinguish these types of motions from excess of motions that might, for example, be due to excess caffeine.

As another example, sleep manager 712 can determine the irregularity (or variability) of the heart rate and respiration rate to determine the person is in an REM sleep state, and, thereby, can generate a signal indicating the stage of the sleep is an REM sleep states. During REM sleep, a heart rate and/or the respiration rate of the user can be described as irregular or with sufficient variability to identify that a user is REM sleep. Thus, the variability of the heart rate and/or respiration rate can be used to determine physiological sleep state of the user. In some examples the irregularity of the heart rate and/or the respiration rate can include any heart rate or respiration rate that varies by more than 5%. In some other cases, the variability of the heart rate and/or the respiration rate can vary by any amounts up from 10% to 15%. These percentages are merely examples and are not intended to be limiting, and ordinarily skilled artisan will appreciate that the tolerances for variable heart rates and respiration rates may be based on user characteristics, such as age, level fitness, gender and the like. Sleep manager 712 can use motion data 705 to confirm whether a user is in an REM sleep state by detecting indicative amounts of motion, such as a portion of motion that includes negligible to no motion. As caffeine may affect the heart rate, consumable correlator 722 is configured to analyze heart rates during different stages of sleep at different levels of caffeine determine whether there is correlation between sleep and caffeine.

Sleep manager 712 is shown to include sleep predictor 714, which is configured to predict the onset or change into or between different stages of sleep. The user may not perceive such changes between sleep states, such as transitioning from a wakefulness state to a sleep state. Sleep predictor 714 can detect this transition from a wakefulness state to a sleep state, as depicted as transition 730. Consumable correlator 722 analyzes these transitions to determine the role whether caffeine actually disrupts the timing of such transitions. Transition 730 may be determined by sleep predictor 740 based on the transitions from irregular heart rate and respiration rates during wakefulness to more regular heart rates and respiration rates during early sleep stages. Also, lowered amounts of motion can also indicate transition 730. In some embodiments, motion data 705 includes a velocity or rate of speed at which a user is traveling, such as an automobile. Upon detecting an impending transition from a wakefulness state into a sleep state, sleep predictor 714 generates an alert signal, such as a vibratory initiation signal, configuring to generate a vibration (or any other response) to convey to a user that he or she is about to fall asleep. So if the user is driving, predictor 714 assists in maintaining a wakefulness state during which the user can avoid falling asleep behind the wheel. If caffeine levels are detected to be low in such situations, the notification engine may generate a notification to consume coffee to increase awareness Sleep predictor 714 can be configured to also detect transition 732 from a light sleep state to a deep sleep state and a transition 734 from a deep sleep state to an REM sleep state. In some embodiments, transitions 732 in 734 can be determined by detected changes from regular to variable heart rates or respiration rates, in the case of transition 734. Also, transition 734 can be described by a decreased level of motion to about zero during the REM sleep state. Further, sleep predictor 714 can be configured to predict a sleep stage transition to disable an alert, such as wake-up time alarm, that coincides with a state of REM sleep. By delaying generation of an alarm, the user is permitted to complete of a state of REM sleep to enhance the quality of sleep.

Controller 702 and one or more constituent elements, such as sleep manager 712, consumable controller 720, and/or consumable correlator 722 can be configured to perform the following. For example, controller 702 can be configured to characterize one or more values of a modified amount at one or more time points, and then correlate one or more characteristics of sleep to a value of the modified amount to form at least one correlated sleep characteristic. Based on the correlation, controller 702, or the like, can determine a quality state of sleep based on the correlated sleep characteristics. For example, relatively high quality states of sleep generally have relatively low disturbances, low occurrences of relatively long times to fall asleep, etc. In some cases, controller 702 can identify an elapsed time to onset of sleep, such as a duration of time between laying down in bed and falling asleep. Further, controller 702; can determine an association between the elapsed time to the onset of sleep and the value of the modified amount of the aggregated amount of the portions of the consumable. With this correlation, recommendations to modify caffeine intake, as a consumable, is possible.

In some cases, controller 702 can determine values characterizing the quality of deep sleep and light sleep. Also, controller 702 can determine one or more associations between either the value characterizing the quality of deep sleep or the value characterizing the quality of light sleep, or both, and the value of the modified amount of the aggregated amount of the portions of the consumable. Upon determining such an association, caffeine intake and some portions of sleep can be correlated. A controller 702 can also determine a value representing the disruptive effect of one or more durations of wakefulness during the sleep activity. By doing so, controller 702 can determine an association between the value representative of the one or more durations of wakefulness and the value of the modified amount of the aggregated amount of the portions of the consumable. Upon determining such an association, caffeine intake and some portions of sleep can be correlated. Further, controller 702 can also determine a value characterizing an amount of motion during the sleep activity to determine whether that motion is not indicative of restful sleep. For example, controller 702 can determine determining an association between an amount of motion during sleep and a value of the modified amount of the aggregated amount of the portions of the consumable.

According to some examples, activity-related managers can include a nutrition manager, a sleep manager, an activity manager, a sedentary activity manager, and the like, examples of which can be found in U.S. patent application Ser. No. 13/433,204, filed on Mar. 28, 2012 having Attorney Docket No. ALI-013CIP1, U.S. patent application Ser. No. 13/433,208, filed Mar. 28, 2012 having Attorney Docket No. ALI-013CIP2, U.S. patent application Ser. No. 13/433,208, filed Mar. 28, 2012 having Attorney Docket No. ALI-013CIP3, U.S. patent application Ser. No. 13/454,040, filed Apr. 23, 2012 having Attorney Docket No. ALI-013CIP1CIP1, U.S. patent application Ser. No. 13/627,997, filed Sep. 26, 2012 having Attorney Docket No. ALI-100, all of which are incorporated herein by reference for all purposes.

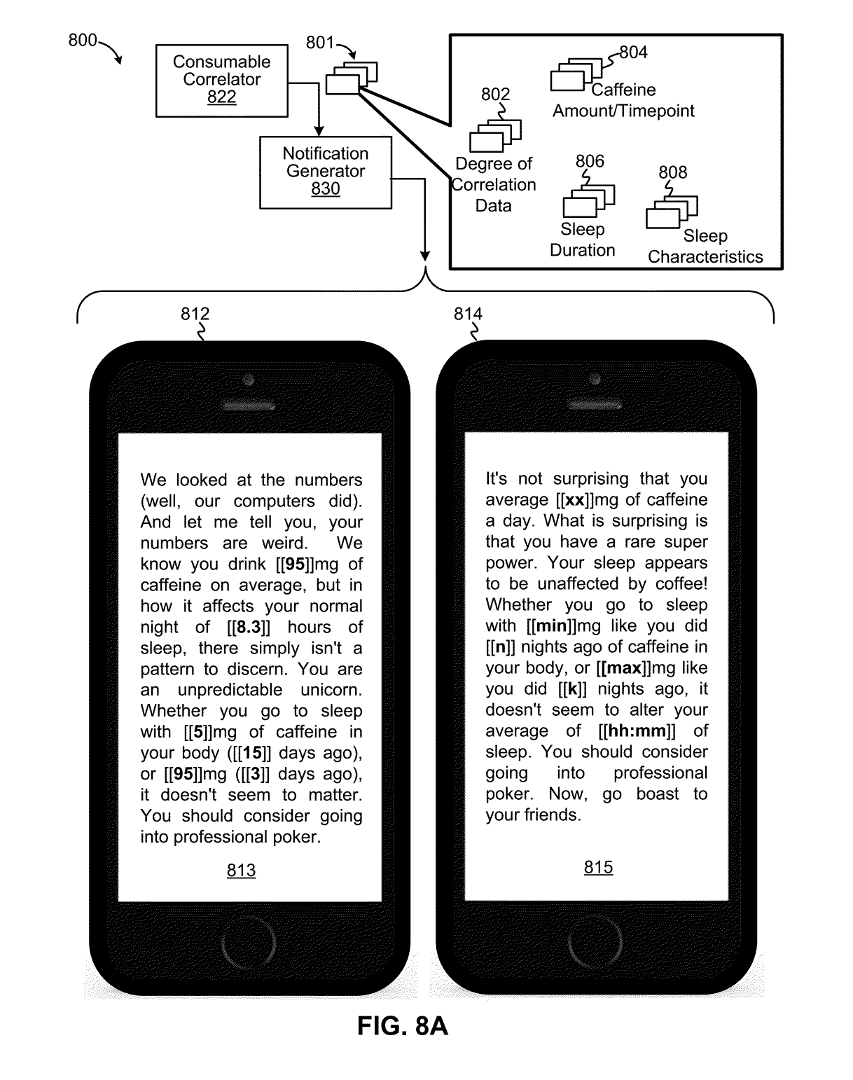

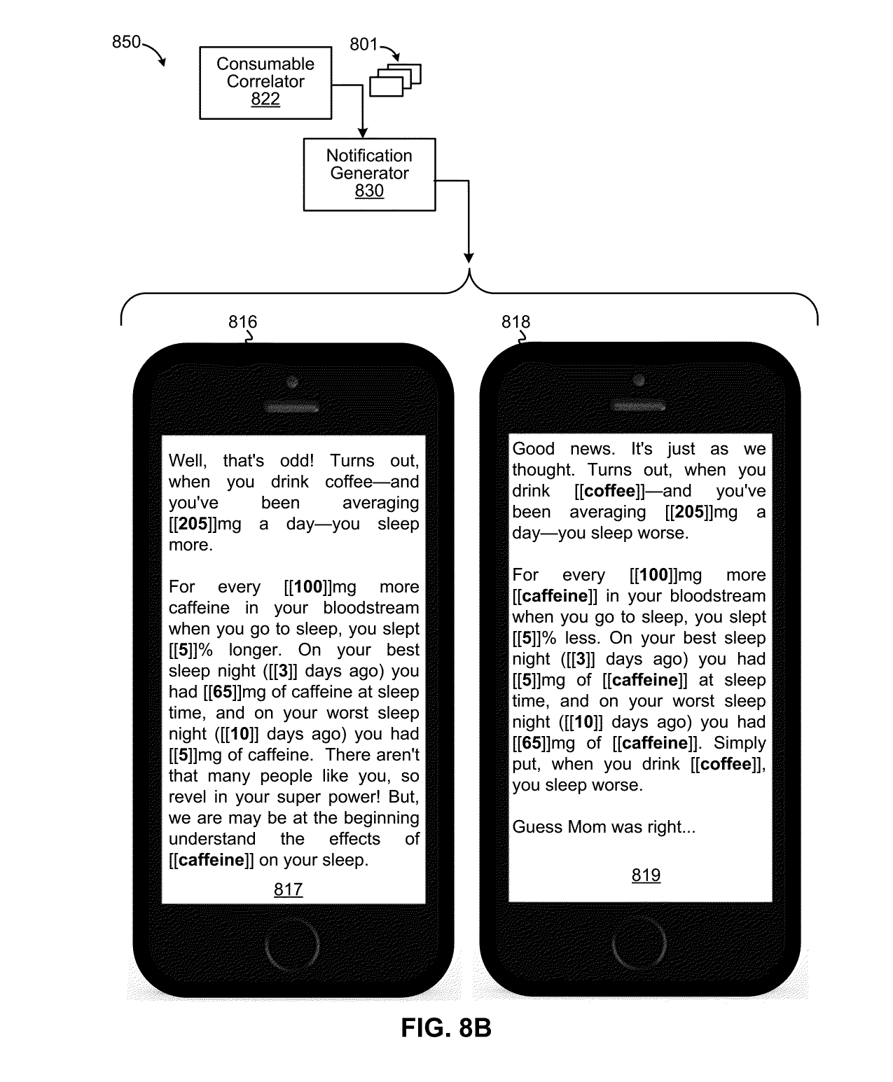

FIGS. 8A and 8B depict a notification generator configured to generate notifications as a function of correlation of data, according to some examples. Diagram 800 of FIG. 8A is shown to include a consumable correlator 822 configured to transmit correlation data 801 to a notification generator 830, which is configured to generate various notifications regarding a user's consumption of a consumable portion, such as caffeine, and a degree of correlation of caffeine to the user's performance of an activity, such as a sleep activity. As shown, correlation data 801 includes data 802 representing a degree of correlation, data 804 representing collection of caffeine amounts relative to multiple time points, data 806 includes data representing the duration of sleep, such as an average duration of sleep, and data 808 includes data representing various sleep characteristics, such as number and length of disruptions during sleep, number and length of deep sleep cycles, number and length of light sleep cycles, the length of time to fall asleep, etc. Examples of caffeine amounts relative to multiple time points include an amount at a predicted time at which a user wishes to go to sleep, an amount of caffeine at the onset of sleep, an minimum amount of caffeine at the onset of sleep, an average amount of caffeine at the onset of sleep, average total caffeine consumption per day, per part of day (e.g., morning, afternoon, evening), per hour, or in view of any other portion of time.

At least in some embodiments, data 802 describing a degree of correlation includes data specifying that relationships between sleep characteristics and caffeine are inconclusive, data specifying that no correlation has been determined between sleep characteristics and caffeine, data specifying a positive correlation between sleep and caffeine, and data specifying a negative correlation between sleep and caffeine. As shown in FIG. 8A, interface 813 of mobile device 812 is presenting an example of a notification or report summarizing that inability to determine whether consumption of a caffeine can be correlated to sleep. Interface 815 of mobile device 814 is presenting an example of a notification or report summarizing that there is no correlation between caffeine intake and sleep. Note that the bracketed values are values determined by, for example, controller 702 of FIG. 7, or by other functions or structures described herein.

Diagram 850 of FIG. 8B depicts an interface 817 of mobile device 816 presenting a notification or report summarizing a positive correlation between consumption of caffeine and sleep, whereas interface 819 of mobile device 818 is presenting a notification or report summarizing a negative correlation between consumption of caffeine and sleep.

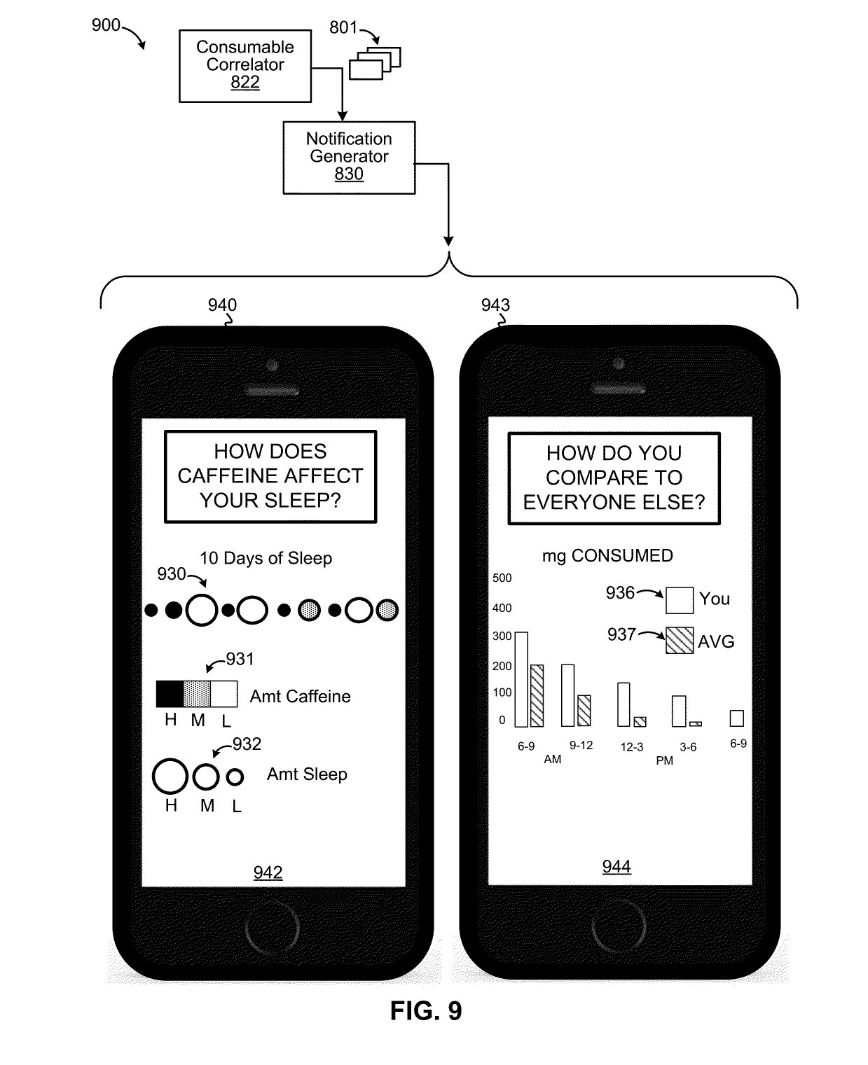

FIG. 9 depicts a notification generator configured to generate reports as a function of correlation of data, according to some examples. Diagram 900 of FIG. 9 is shown to include a consumable correlator 822 configured to transmit correlation data 801 to a notification generator 830, which is configured to generate various notifications regarding a user's consumption of a consumable portion, such as caffeine, and a degree of correlation of caffeine to the user's performance of an activity, such as a sleep activity.

Interface 942 of mobile device 940 is shown to present a graphical summary correlating amounts of caffeine consumed against amounts of sleep. A duration 903 depicting graphical representations of 10 days of sleep, whereby each day is represented by a circle having a size relative to the amount of sleep for that day, and a degree of shading relative to the amount of caffeine consumed for that day. By contrast, interface 944 of mobile device 943 is shown to present a graphical summary correlating amounts of caffeine 936 consumed by the user against amounts consumed 937, on average, by either that person over a historical period of time or in view of a group of colleagues or cohorts.

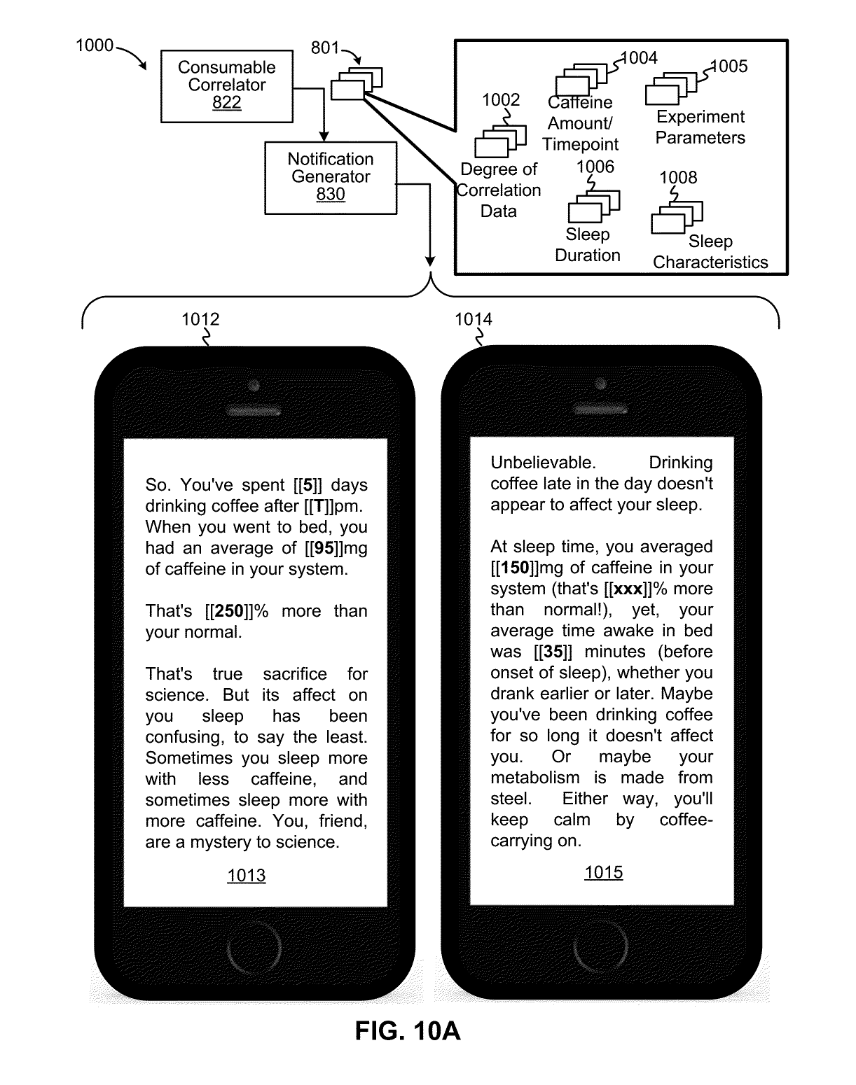

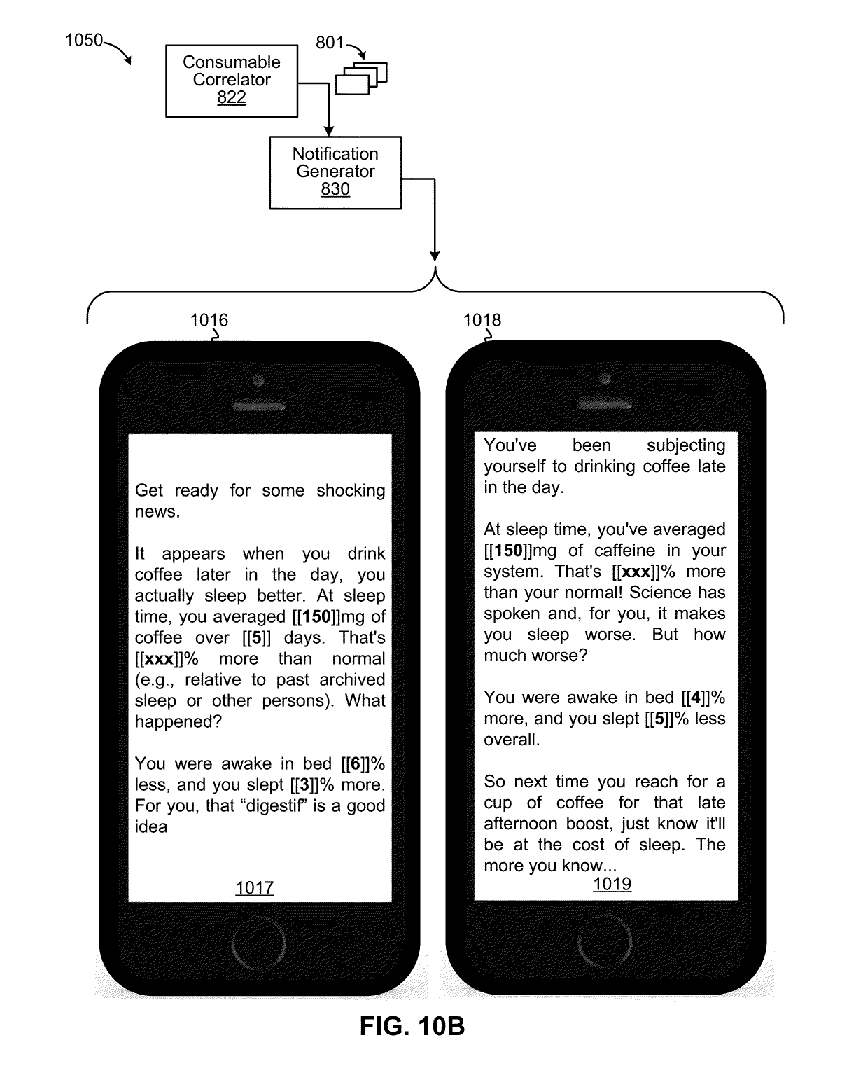

FIGS. 10A and 10B depict a notification generator configured to generate notifications as a function of correlation of data, according to some examples. Diagram 1000 of FIG. 10A is shown to include a consumable correlator 822 configured to transmit correlation data 801 to a notification generator 830, which is configured to generate various notifications regarding a user's consumption of a consumable portion, such as caffeine, and a degree of correlation of caffeine to the user's performance of an activity, such as a sleep activity.

In at least this example, amounts of caffeine consumed after 5 PM are analyzed against sleep characteristics to determine whether caffeine consumption late in the day affects the user sleep cycle. As shown, correlation data 801 includes data 1002 representing a degree of correlation, data 1004 representing collection of caffeine amounts relative to multiple time points, data 1006 includes data representing the duration of sleep, data 1005 includes data representing experiment parameters (i.e., parameters in which to generate a report limited to certain correlations, such as correlating a time of day when caffeine is consumed and subsequent sleep), and data 1008 includes data representing various sleep characteristics, as described above.

At least in some embodiments, data 1002 describing a degree of correlation includes data specifying that relationships between sleep characteristics and caffeine are inconclusive, data specifying that no correlation has been determined between sleep characteristics and caffeine, data specifying a positive correlation between sleep and caffeine, and data specifying a negative correlation between sleep and caffeine. As shown in FIG. 10A, interface 1013 of mobile device 1012 is presenting an example of a notification or report summarizing that inability to determine whether consumption of caffeine after 5 PM can be correlated to sleep. Interface 1015 of mobile device 1014 is presenting an example of a notification or report summarizing that there is no correlation between caffeine intake after 5 PM and sleep. Note that the bracketed values are values determined by, for example, controller 702 of FIG. 7, or by other functions or structures described herein.

Diagram 1050 of FIG. 10B depicts an interface 1017 of mobile device 1016 presenting a notification or report summarizing a positive correlation between consumption of caffeine after 5 PM and sleep, whereas interface 1019 of mobile device 1018 is presenting a notification or report summarizing a negative correlation between consumption of caffeine after 5 PM and sleep.

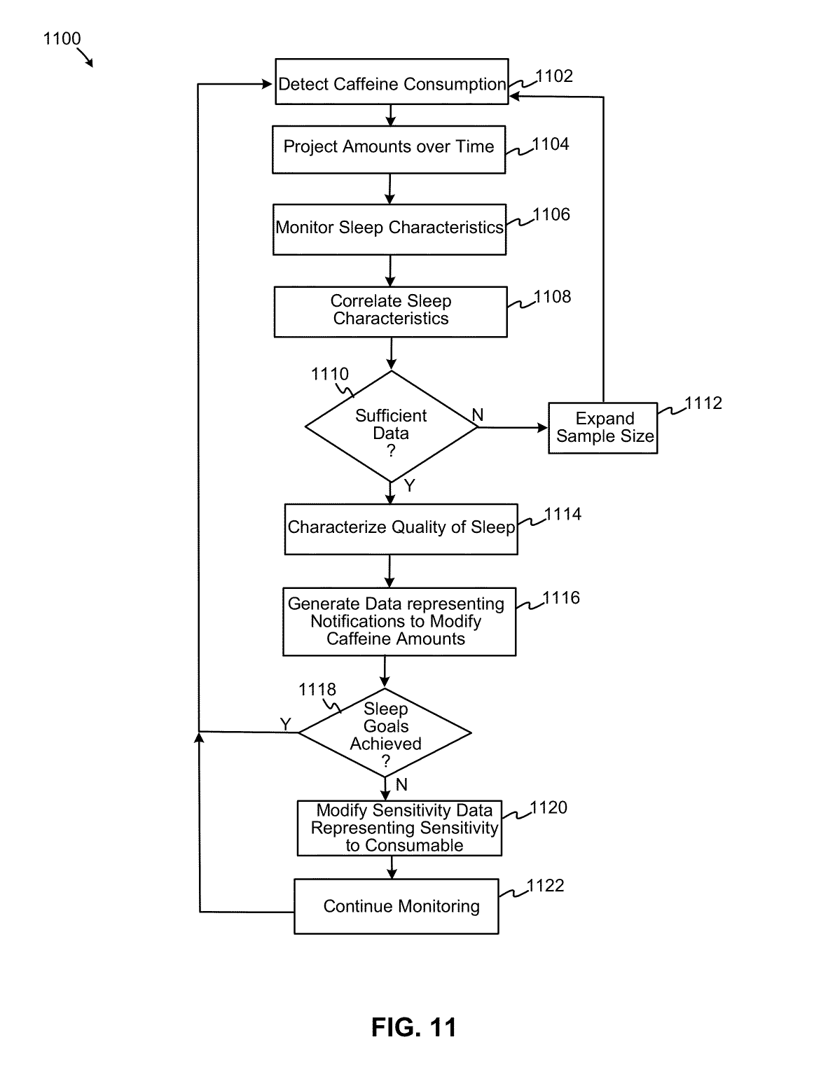

FIG. 11 is a flowchart for correlating sleep characteristics and consumption of a consumable, such as caffeine, according to some embodiments. At 1102, an amount of caffeine consumed (or to be consumed) is detected. At 1104, various modified amounts of caffeine are projected or predicted over time, for different time points, as caffeine is projected to metabolize at a specific rate. At 1106, sleep characteristics are monitored, with those sleep characteristics being correlated with caffeine consumption at 1108. Determination is made at 1110 as to whether sufficient data has been gathered to correlate sleep characteristics and caffeine intake. If not, flow 1100 moves to 1112 at which a sample size is expanded to include more data samples. If so, flow 1100 moves to 1114 to characterize the quality of sleep. In some instances, data is generated at 1116 to provide notifications to a user to modify caffeine amounts (e.g., refrain from drinking coffee after a certain time). If sleep goals are achieved at 1118, further caffeine consumption is monitor, otherwise flow 1100 moves to 1120 to modify sensitivity data that represents a user's sensitivity to a consumable. For example, a user may underestimate their sensitivity to caffeine and continue to consume more than what is optimal to achieve their sleeping goals. At 1122, monitoring caffeine consumption continues.

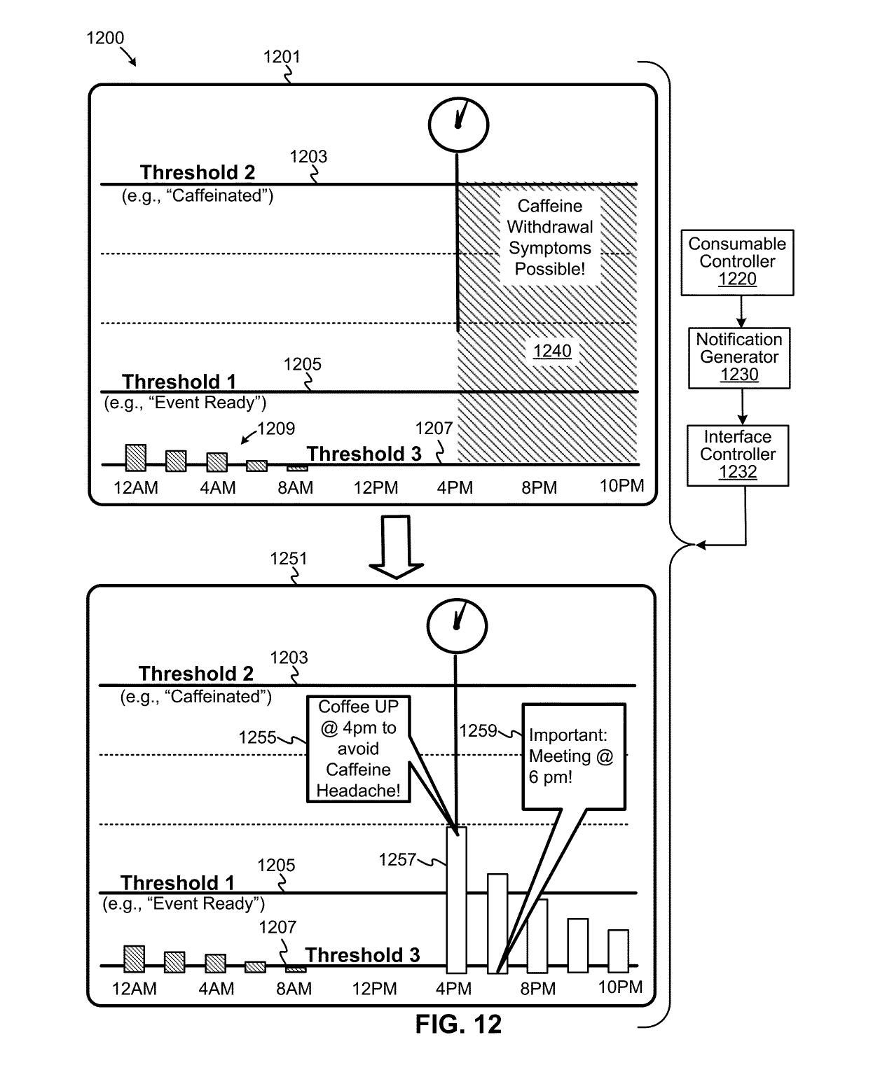

FIG. 12 is a block diagram depicting generation of a notification to avoid symptoms associated with withdrawal, according to some examples. Diagram 1200 depicts a consumable controller 1220 coupled to a notification generated 1230, which, in turn, is coupled to interface controller 1232. Interface controller 1232 generates interface 1201 depicting a first threshold 1205 and a second threshold 1203, both of which are similar in function to those thresholds described above. Further, interface controller 1232 is configured to display a graphical representation of a threshold 1207, which depicts a level below which a reduced caffeine level for an extended period of time may lead to caffeine withdrawal symptoms, which includes headaches, shakiness, etc. In some examples, consumable controller 1220 is configured to determine a duration in an aggregated amount of caffeine (or aggregated amounts of consumable portions) that falls below a threshold representing a window of time in which caffeine withdrawal symptoms arise. Consumable controller 1220 is configured to monitor a lack of caffeine intake for the previous several hours, whereby the caffeine levels 1209 reflect a diminishing amount in a user's system. In response, consumable controller 1220 causes notification generator 1230 to generate a warning 1240. Further, notification generator 1230 operates to modify interface 1201 to appear as interface 1251 in which notifications are generated for the user suggesting to consume coffee via a notification 1255 in view of a notification 1259, which indicates an impending meeting is few hours away. Notification 1255 suggests that the user drink amount 1257 of coffee so as to avoid painful withdrawal symptoms might otherwise interfere with the user's performance in a meeting.

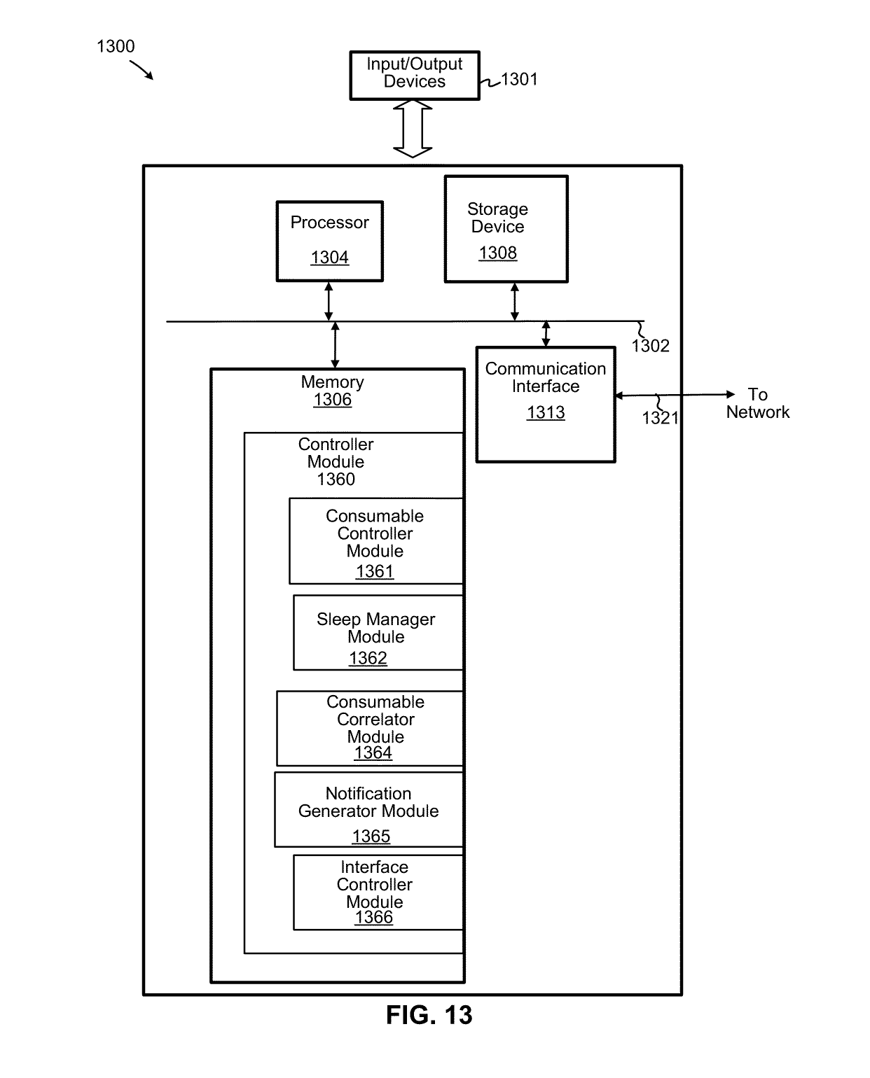

FIG. 13 illustrates an exemplary computing platform in accordance with various embodiments. In some examples, computing platform 1300 may be used to implement computer programs, applications, methods, processes, algorithms, or other software to perform the above-described techniques. Computing platform 1300 includes a bus 1302 or other communication mechanism for communicating information, which interconnects subsystems and devices, such as processor 1304, system memory 1306 (e.g., RAM, etc.), storage device 1308 (e.g., ROM, etc.), a communication interface 1313 (e.g., an Ethernet or wireless controller, a Bluetooth controller, etc.) to facilitate communications via a port on communication link 1321 to communicate, for example, with a computing device, including mobile computing and/or communication devices with processors. Processor 1304 can be implemented with one or more central processing units (“CPUs”), such as those manufactured by Intel® Corporation, or one or more virtual processors, as well as any combination of CPUs and virtual processors. Computing platform 1300 exchanges data representing inputs and outputs via input-and-output devices 1301, including, but not limited to, keyboards, mice, audio inputs (e.g., speech-to-text devices), user interfaces, displays, monitors, cursors, touch-sensitive displays, LCD or LED displays, and other I/O-related devices. An interface is not limited to a touch-sensitive screen and can be any graphic user interface, any auditory interface, any haptic interface, any combination thereof, and the like.

According to some examples, computing platform 1300 performs specific operations by processor 1304 executing one or more sequences of one or more instructions stored in system memory 1306, and computing platform 1300 can be implemented in a client-server arrangement, peer-to-peer arrangement, or as any mobile computing device, including smart phones and the like. Such instructions or data may be read into system memory 1306 from another computer readable medium, such as storage device 1308. In some examples, hard-wired circuitry may be used in place of or in combination with software instructions for implementation. Instructions may be embedded in software or firmware. The term “computer readable medium” refers to any tangible medium that participates in providing instructions to processor 1304 for execution. Such a medium may take many forms, including but not limited to, non-volatile media and volatile media. Non-volatile media includes, for example, optical or magnetic disks and the like. Volatile media includes dynamic memory, such as system memory 1306.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1302 for transmitting a computer data signal.

In some examples, execution of the sequences of instructions may be performed by computing platform 1300. According to some examples, computing platform 1300 can be coupled by communication link 1321 (e.g., a wired network, such as LAN, PSTN, or any wireless network) to any other processor to perform the sequence of instructions in coordination with (or asynchronous to) one another. Computing platform 1300 may transmit and receive messages, data, and instructions, including program code (e.g., application code) through communication link 1321 and communication interface 1313. Received program code may be executed by processor 1304 as it is received, and/or stored in memory 1306 or other non-volatile storage for later execution.

In the example shown, system memory 1306 can include various modules that include executable instructions to implement functionalities described herein. In the example shown, system memory 1306 includes a controller module 1360, which, in turn, includes a consumable controller module 1361, a sleep manager module 1362, a consumable correlator module 1364, a notification generator module 1365, and an interface controller module 1366.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A method comprising:
receiving data representing an indication of consumption of a consumable;
identifying an amount of a portion of the consumable;
receiving data representing a time of an event;
aggregating the amount of the portion of the consumable and a cumulative amount of the portions of the consumable to form an aggregated amount of the portions of the consumable;
determining a modified amount of the aggregated amount of the portions of the consumable as a function of a dissipation rate;
predicting the modified amount at the time of the event to form a predicted amount; and
causing presentation of a representation of a time at which the predicted amount conforms to the threshold value.

2. The method of claim 1, further comprising:
generating data representing a notification configured to cause the predicted amount of the portion of the consumable to conform to the threshold value at the time of the event.

3. The method of claim 1, wherein identifying the amount of the portion of the consumable comprises:
identifying an amount of caffeine.

4. The method of claim 1, further comprising:
generating data representing a representation of a number of units of the aggregated amount of the portions of the consumable; and
causing presentation of the representation of the number of units.

5. The method of claim 4, further comprising:
reducing the number of units as a function of the dissipation rate to form a reduced number of units of the aggregated amount; and
causing presentation of a representation of the reduced number of units.

6. The method of claim 1, further comprising:
monitoring characteristics of an activity

7. The method of claim 6, wherein monitoring the characteristics of the activity comprises:
monitoring characteristics of a sleep activity.

8. The method of claim 7, further comprising:
characterizing one or more values of the modified amount at one or more time points;
correlating one or more characteristics of the sleep activity to a value of the modified amount to form correlated sleep characteristics; and
determining a quality state of sleep based on the correlated sleep characteristics.

9. The method of claim 8, wherein the value of the modified amount coincides with a start time for the sleep activity.

10. The method of claim 8, wherein correlating the one or more characteristics of the sleep activity comprises:
identifying an elapsed time to onset of sleep; and
determining an association between the elapsed time to the onset of sleep and the value of the modified amount of the aggregated amount of the portions of the consumable.

11. The method of claim 8, wherein correlating the one or more characteristics of the sleep activity comprises:
determining a value characterizing a quality of deep sleep;
determining a value characterizing a quality of light sleep; and
determining one or more associations between either the value characterizing the quality of deep sleep or the value characterizing the quality of light sleep, or both, and the value of the modified amount of the aggregated amount of the portions of the consumable.

12. The method of claim 8, wherein correlating the one or more characteristics of the sleep activity comprises:
determining a value representative of a disruptive effect of one or more durations of wakefulness during the sleep activity;
determining an association between the value representative of the one or more durations of wakefulness and the value of the modified amount of the aggregated amount of the portions of the consumable.

13. The method of claim 8, wherein correlating the one or more characteristics of the sleep activity comprises:
determining a value characterizing an amount of motion during the sleep activity; and
determining an association between the amount of motion during the sleep activity and the value of the modified amount of the aggregated amount of the portions of the consumable.

14. The method of claim 1, further comprising:
generating data representing different values of the modified amount at different time points; and
causing presentation of the representation of the different values at the different time points.

15. The method of claim 1, further comprising:
generating data representing a representation of a state of the user as a function of the modified amount; and
causing presentation of the representation of the state of the user.

16. The method of claim 1, wherein receiving the data representing the indication comprises:
receiving data representing a time at which the consumable is consumed.

17. The method of claim 1, further comprising:
modifying sensitivity data representing sensitivity to the portion of the consumable.

18. The method of claim 1, further comprising:
determining a duration in the aggregated amount of the portions of the consumable falls below another threshold representing a window of time in which caffeine withdrawal symptoms arise,
wherein the portions of the consumable includes caffeine.

19. A device comprising:
a memory including executable instructions; and
a processor configured to:
execute a first portion of the executable instructions to receive data representing an indication of consumption of a consumable;
execute a second portion of the executable instructions to identify an amount of caffeine;
execute a third portion of the executable instructions to aggregate the amount of caffeine with a cumulative amount of caffeine to form an aggregated amount of caffeine of the consumable;
execute a fourth portion of the executable instructions to determine a modified amount of the aggregated amount of caffeine of the consumable as a function of a dissipation rate;
execute a fifth portion of the executable instructions to predict the modified amount at the time of the event to form a predicted amount; and
execute a sixth portion of the executable instructions to cause presentation of a representation of a time at which the predicted amount conforms to the threshold value,
wherein the presentation is to be displayed on an interface.

20. The device of claim 19, further comprising:
executable instructions to characterize one or more values of the modified amount at one or more time points;
executable instructions to correlate one or more characteristics of the sleep activity to a value of the modified amount to form correlated sleep characteristics; and
executable instructions to determine a quality state of sleep based on the correlated sleep characteristics.
